(19) United States
(12) Patent Application Publication
(10) Pub. No.: US 2025/0044273 A1
(43) Pub. Date: Feb. 6, 2025

(54) CONTROL OF OXIDIZING BIOCIDE REAGENTS TO MITIGATE BIOFOULING

(71) Applicant: ECOLAB USA Inc., St. Paul, MN (US)

(72) Inventors: Ning JIN, Shanghai (CN); Seong Hoon YOON, Naperville, IL (US); Harshada Ramesh LOHOKARE, Pune (IN)

(73) Assignee: ECOLAB USA Inc., St. Paul, MN (US)

(21) Appl. No.: 18/784,203

(22) Filed: Jul. 25, 2024

Related U.S. Application Data
(60) Provisional application No. 63/516,627, filed on Jul. 31, 2023.

(51) Int. Cl. G01N 33/18; C02F 1/76

(52) U.S. Cl. CPC G01N 33/18; C02F 1/766; C02F 2303/20

(57) ABSTRACT

The present disclosure provides methods of monitoring and controlling biocides in an aqueous medium, methods of manufacturing the compositions, and various oxidizing biocide compositions suitable for use in aqueous medium treatment processes. Methods include adding a first oxidizing biocide at a first injection point upstream of a reverse osmosis (RO) membrane inlet, adding a second oxidizing biocide at a second injection point upstream of the RO membrane inlet, obtaining a first sample of the aqueous medium from a first location downstream of the first and second injection points and upstream of the RO membrane inlet, and obtaining a second sample of the aqueous medium from a second location downstream of the first location but immediately upstream of the RO membrane. Methods may also include measuring free residual oxidant concentration, total residual oxidant concentration, and oxidation-reduction potential of the first and second samples and adjusting biocide addition based on the measurements.

### BACKGROUND

Biological fouling, or biofouling, is the accumulation of microorganisms, viscous liquids, and impurities on wet surfaces, such as membranes of membrane separation devices (e.g., a reverse osmosis (RO) membrane or nanofiltration membrane separation device) used to treat water. Biofouling of such membranes negatively impacts the membrane osmotic pressure. Biofouling also reduces the flow rate and quality of the water produced and increases the operation pressure and overall pressure drop of the system, which negatively affects the device operation. While cleaning chemicals are applied during device operation to mitigate biofouling, the negative operation impacts of biofouling will likely nonetheless reduce the service life of the membrane.

### BRIEF SUMMARY

According to one or more embodiments, methods of monitoring and controlling biocides in an aqueous medium include adding a first oxidizing biocide at a first injection point upstream of a reverse osmosis (RO) membrane inlet. The methods further include adding a second oxidizing biocide at a second injection point upstream of the RO membrane inlet. The methods also include obtaining a first sample of the aqueous medium from a first location downstream of the first and second injection points and upstream of the RO membrane inlet, obtaining a second sample of the aqueous medium from a second location downstream of the first location but upstream of the RO membrane inlet, measuring a free residual oxidant concentration of the first sample and the second sample using a reagent comprising N,N-diethyl-p-phenylenediamine (DPD), and adjusting addition of the first and/or second oxidizing biocide so that the free residual oxidant concentration in the second sample is less than about 0.1 mg/L as Cl₂ and the free residual oxidant concentration in the first sample is higher than about 0.1 mg/L as Cl₂.

In other embodiments, methods of monitoring and controlling biocides in an aqueous medium include adding a first oxidizing biocide at a first injection point upstream of a RO membrane inlet. The methods further include adding a second oxidizing biocide at a second injection point upstream of the RO membrane inlet. The methods also include obtaining a first sample of the aqueous medium from a first location downstream of the first and second injection points and upstream of the RO membrane inlet, and obtaining a second sample of the aqueous medium from a second location downstream of the first location and upstream of the RO membrane inlet. The methods also include measuring a total residual oxidant concentration of the first sample using a reagent comprising DPD, measuring a total residual oxidant concentration of the second sample using the reagent comprising DPD, and adjusting biocide addition based on a difference between the measured total residual oxidant concentration at the second location and the measured total residual oxidant concentration at the first location.

Yet, in other embodiments, methods of monitoring and controlling biocides in wastewater include adding a first oxidizing biocide at a first injection point upstream of a RO membrane inlet, and adding a second oxidizing biocide at a second injection point upstream of the RO membrane inlet. The methods further include measuring a first oxidation-reduction potential (ORP) at a first location downstream of the first and second injection points but upstream of the RO membrane inlet, and measuring a second ORP from a second location downstream of the first location and upstream of the RO membrane inlet and adjusting the amount of the first and/or second oxidizing biocide being added based on the difference of the first and second ORP and if the second ORP is greater or less than about 300 mV.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### BRIEF DESCRIPTION OF DRAWINGS

For a more complete understanding of this disclosure, reference is now made to the following brief description, taken in connection with the accompanying drawings and detailed description, wherein like reference numerals represent like parts.

FIG. 1 illustrates a process flow of methods of embodiments of the present disclosure.

FIG. 2 illustrates a graph demonstrating total viable count as a function of various biocides.

FIG. 3 illustrates a graph of consumption of biocides as a function of time.

FIG. 4 illustrates a graph of consumption of biocides as a function of time.

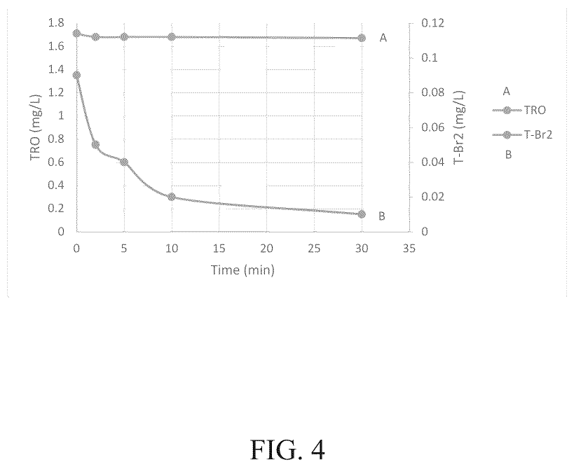

FIG. 5 illustrates a process flow of methods of embodiments of the present disclosure.

### DETAILED DESCRIPTION

Although the present disclosure provides references to various embodiments, persons skilled in the art will recognize that changes may be made in form and detail without departing from the spirit and scope of the disclosure. Additionally, any examples set forth in this specification are not intended to be limiting and merely set forth some of the many possible embodiments for the appended claims.

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present invention. All publications, patent applications, patents and other references mentioned herein are incorporated by reference in their entirety.

As used herein, the terms “comprise(s),” “include(s),” “having,” “has,” “can,” “contain(s),” and variants thereof are intended to be open-ended transitional phrases, terms, or words that do not preclude the possibility of additional acts or structures. The singular forms “a,” “and” and “the” include plural references unless the context clearly dictates otherwise.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

As used herein, the term “optional” or “optionally” means that the subsequently described event or circumstance may but need not occur, and that the description includes instances where the event or circumstance occurs and instances in which it does not.

As used herein, the term “about” modifying, for example, the quantity of an ingredient in a composition, concentration, volume, process temperature, process time, yield, flow rate, pressure, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities. The term “about” refers to the cited value being within the errors arising from the standard deviation found in their respective testing measurements, and if those errors cannot be determined, then “about” may refer to, for example, within 5% of the cited value.

Further, where “about” is employed to describe a range of values, for example “about 1 to 5,” the recitation means “1 to 5” and “about 1 to about 5” and “1 to about 5” and “about 1 to 5,” unless specifically limited by context.

A composition that is “substantially free” of a specified compound or material may be free of that compound or material, or may have a minor amount of that compound or material present, such as through unintended contamination, side reactions, incomplete purification or test methods used. A “minor amount” may be a trace, an unmeasurable amount, an amount that does not interfere with a value or property, or some other amount as provided in context. A composition that has “substantially only” a provided list of components may consist of only those components, or have a trace amount of some other component present, or have one or more additional components that do not materially affect the properties of the composition. Additionally, “substantially” modifying, for example, the type or quantity of an ingredient in a composition, a property, a measurable quantity, a method, a value, or a range, employed in describing the embodiments of the disclosure, refers to a variation that does not affect the overall recited composition, property, quantity, method, value, or range thereof in a manner that negates an intended composition, property, quantity, method, value, or range. Where modified by the term “substantially,” the claims appended hereto include equivalents according to this definition.

As used herein, any recited ranges of values contemplate all values within the range and are to be construed as support for claims reciting any sub-ranges having endpoints which are real number values within the recited range. By way of example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

Unless specified otherwise, all molecular weights referred to herein are weight average molecular weights and all viscosities were measured at about 25° C. with neat (not diluted) polymers.

Microbial fouling of RO membranes is a challenge for wastewater recycle systems. While halogen based oxidizing biocides, such as sodium hypochlorite (NaClO), are used to mitigate biofouling in water treatment devices, they are generally not used to treat RO systems because the most widely used RO membranes, polyamide (PA) thin film composite (TFC) membranes, are susceptible to oxidative damaged by halogens. If halogens are present in the influent water, persistent exposure ultimately results in loss of membrane lifetime, as measured by increased passage of both salt and water.

Nonetheless, stabilized chlorine reagents can be used to treatment RO processes. When dosed or added into feed water, stabilized chlorine reagents release a small amount of active chlorine species, such as hypochlorous acid (HOCl). Such chlorine species can effectively eliminate microorganisms, and further, due to their very low concentration, their damaging effects to RO membranes are weak enough such that they do not induce significant membrane damage.

On the contrary, one challenge with stabilized chlorine-based biocides is that they have very limited biocidal effectiveness. In particular, these biocides can only maintain a biostatic environment and prevent further bacterial growth. In wastewater recycle systems where microbial growth potential is generally high, stabilized chlorine-based biocides have limited applications because they often do not have sufficient biocidal power to decrease microbes to desirable levels.

In contrast to chlorine-based reagents, bromine-based reagents have significantly greater biocidal effectiveness. Despite their increased effectiveness, one downside is that the resulting bromine species cause significantly faster damage to RO membranes. As such, even in stabilized forms that result in slower release of damaging bromine species, bromine-based reagents nonetheless induce too much damage to be generally compatible with RO membranes. Even trace amounts of bromine species generated in situ can induce damage RO membranes if the surrounding pH level is low.

Accordingly, described herein are biocidal reagents and methods of making and using thereof that provide effective biocidal effectiveness in aqueous media without damaging RO membranes, resulting in advantages of increased device lifetimes and stability. Also disclosed are methods of monitoring and controlling biocides in an aqueous medium. The aqueous medium includes water. In one or more embodiments, the aqueous medium includes wastewater. In embodiments, the aqueous medium is in or processed in a membrane separation device. Non-limiting examples of the membranes in the membrane separation device include a RO membrane, a nanofiltration membrane (NF), an ultrafiltration membrane (UF), a microfiltration membrane (MF), an electrodialysis (ED), or any combination thereof. Non-limiting examples of the membrane separation device include a RO membrane filtration device, a NF separation device, an UF separation device, a MF separation device, an ED separation device, or any combination thereof. The form of the filtration membrane and device is not limited, and any type of membrane module, such as spiral wound type membrane module, hollow-fiber membrane module, tubular type membrane module, plane type membrane module, or any combination thereof, may be used.

Non-limiting examples of materials for the membrane polymers made from nitrogen-containing groups, such as aromatic polyamides, polyurea, polypiperazine-amide, or any combination thereof.

Such membrane separation devices can be used to the field of water treatment and can be used for preparation of other types of water, such as drinking water, pure water, ultra-pure grade water, process water for electricity generation, electronic method process water, semiconductor method process water, process water for medical field applications, water for chemical or biological agents, water for injection, aseptic pyrogen-free pure water, process water for food and beverage applications, chemical engineering and other engineering process water, water for boiler applications, water for washing and/or cooling, or any combination thereof. Such membrane separation devices can also be applied to the fields such as desalination of seawater or brackish water.

Embodiments include methods for inhibiting the biofouling growth in a membrane separation device for water treatment or removing, decreasing, or mitigating the biofouling on a membrane of a membrane separation device for water treatment. By applying the methods and compositions of the present invention, the growth of biofouling can be effectively inhibited without damaging the filtration membrane itself.

In embodiments, methods include adding a first oxidizing biocide at a first injection point upstream of membrane inlet, such as a RO membrane inlet. In embodiments, the methods also include adding a second oxidizing biocide at a second injection point upstream of the RO membrane inlet.

Non-limiting examples of the first and second oxidizing biocides include chlorosulfamate, bromosulfamate, iodosulfamate, a stabilized chlorine or a stabilized bromine stabilized by ammonia, ethanolamine, or organic sulfamate, or any combination thereof. In embodiments, the first oxidizing biocide is the stabilized bromine. In other embodiments, the second oxidizing biocide is the stabilized chlorine.

According to one or more embodiments, the first oxidizing biocide includes from about 1 mol % to about 50 mol % of a total amount of biocide added. In some embodiments, the first oxidizing biocide includes from about 10 mol % to about 40 mol % of a total amount of biocide added. Yet, in other embodiments, the first oxidizing biocide includes from about 20 mol % to about 30 mol % of a total amount of biocide added. Still yet, in embodiments, the first oxidizing biocide includes about or any range between about 1, 5, 10, 15, 20, 25, 30, 35, 40, 45, and 50 mol % of a total amount of biocide added.

According to one or more embodiments, the second oxidizing biocide includes from about 50 mol % to about 99 mol % of a total amount of biocide added. In some embodiments, the second oxidizing biocide includes from about 60 mol % to about 90 mol % of a total amount of biocide added. Yet, in other embodiments, the second oxidizing biocide includes from about 70 mol % to about 80 mol % of a total amount of biocide added. Still yet, in embodiments, the second oxidizing biocide includes about or any range between about 50, 55, 60, 65, 70, 75, 80, 85, 90, 95, and 99 mol % of a total amount of biocide added.

In one or more embodiments, a total biocide concentration as Cl₂ is between about 10 parts per billion (ppb) and about 100 ppm. In some embodiments, the total biocide concentration as Cl₂ is between about 20 ppb and about 90 ppm. In other embodiments, the total biocide concentration as Cl₂ is between about 30 ppb and about 80 ppm. Yet, in other embodiments, the total biocide concentration as Cl₂ is about or in any range between about 10, 15, 20, 25, 30, 35, 40, 45, 50, 55, 60, 65, 70, 75, 80, 85, 90, and 100 ppb.

According to one or more embodiments, the methods include adding a third biocide at a third injection point upstream of the RO membrane inlet. Non-limiting examples of the third biocide include dibromonitrilopropionamide (DBNPA), an isothiazolinone, such as chloromethylisothiazolinone, chlorosulfamate, bromosulfamate, iodosulfamate, a stabilized chlorine or stabilized bromine stabilized by ammonia, urea, ethanolamine, or organic sulfamate, or any combination thereof.

Any method may be used to prepare the biocides described herein, prior to adding to the injection points.

In embodiments, the first oxidizing biocide is added simultaneously with the second oxidizing biocide, sequentially with the second oxidizing biocide, continuously, and/or intermittently. In one or more embodiments, the second oxidizing biocide is added simultaneously with the first oxidizing biocide, sequentially with the first oxidizing biocide, continuously, and/or intermittently. In some embodiments, the third biocide is added simultaneously with the first and/or second oxidizing biocide, sequentially with the first and/or second oxidizing biocide, continuously, and/or intermittently. In embodiments, the first and second oxidizing biocides are added simultaneously or intermittently and the first and second injection points are at substantially the same location. Yet, in other embodiments, the first and second oxidizing biocides are added simultaneously or intermittently and the first injection point is further from the RO membrane than the second injection point or wherein the second injection point is further from the RO membrane than the first injection point.

When the first oxidizing biocide (e.g., stabilized bromine) and second oxidizing biocide (e.g., stabilized chlorine), optionally, third oxidizing biocide, are dosed into the aqueous medium, they react with organic materials, such natural organic matter (NOM), microorganisms, and their extracellular polymeric substances (EPS).

Inorganic compounds, such as ammonia, sulfite, ferrous, manganese ions, and the like, also react with these oxidizing biocide reagents. In embodiments, an oxidizing halogen of the first oxidizing biocide (e.g., bromine) is more reactive and consumed at a faster rate than an oxidizing halogen of the second oxidizing biocide (e.g., chlorine). The ultimate fate of active oxidizing halogen species of the first and second oxidizing biocides are halides, such as, bromide and chloride ions, respectively. The consumption rate of the first oxidizing biocide halogen, e.g., bromine, in both stabilized and free forms, is dependent on the nature and concentration of organic and inorganic molecules in aqueous medium. The aqueous medium properties, such as pH and temperature and the nature of stabilizers in first and second oxidizing biocides, also play important roles. The complete consumption of the oxidizing halogen species of the first oxidizing biocide can range from nearly instantly (e.g., less than 1 second after mixing) to longer periods of time (e.g., about 1 to about 5 hours). In embodiments, the oxidizing halogen of the first oxidizing biocide is dosed in the upstream processes, such as upstream of filters and/or feeding tanks, to provide the biocidal power, and all of such damaging oxidizing halogen, in both stabilized and free forms, is consumed before it reaches RO membranes.

According to embodiments, the first injection point and/or the second injection point is before a cartridge filter, a feeding tank, an ion-exchange unit, an activated carbon filtration unit, a micro/ultrafiltration unit, and/or a sand filtration unit. According to other embodiments, the methods include consuming the first oxidizing biocide before it reaches the RO membrane inlet.

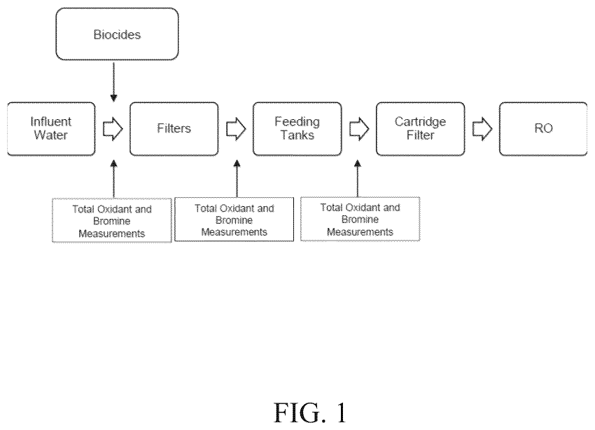

FIG. 1 illustrates a process flow of a method according to embodiments, in which biocides, including one or more of the first oxidizing biocide, the second oxidizing biocide, and the third oxidizing biocide, is injected into influent water of a membrane separation device and before one or more filters, feeding tanks, cartridge filters, and/or RO membranes. As shown, before influent water actually contacts RO membranes, the systems include filtration or polishing processes (“Filters” in FIG. 1), such as sand filtration (SF), micro/ultrafiltration (MF/UF), activated carbon filtration (ACF) and ion-exchange (IX) filter processes. There can be several feeding tanks between the processes (“Feeding Tanks”), and optionally, further a cartridge filter (CF) or safety filter (“Cartridge Filter”) right before RO membrane (“RO”) to protect the same.

In embodiments, the methods include obtaining a first sample of the aqueous medium from a first location downstream of the first and second injection points and upstream of the RO membrane inlet. In other embodiments, the methods further include measuring a biocide concentration of the first sample using a reagent comprising DPD. As shown in FIG. 1, total oxidant and bromine measurements can be used to measure biocide concentration at various points in the system. An initial total oxidant and bromine measurement can be taken at one or more points (first point and second point) after initial biocide injection. Subsequently, the total oxidant and bromine measurements can be taken at one or more downstream points, such as between a filter and feeding tank (or downstream of a filter and upstream of a feeding tank), and/or between a feeding tank and cartridge filter (or downstream of a feeding tank and upstream of a cartridge filter).

In some embodiments, methods include obtaining a second sample of the aqueous medium from a second location downstream of the first location and upstream of the RO membrane inlet, and measuring a biocide concentration of the second sample using the reagent with the DPD. Still, in further embodiments, the methods include adjusting biocide addition based on a difference between the measured biocide concentration at the second location and the measured biocide concentration at the first location.

In embodiments, methods further include measuring a total residual oxidant concentration of the first and second samples; calculating a difference in the total residual oxidant concentrations of the first and second samples; and further reducing an amount of the first and/or second oxidizing biocide being added if the total residual oxidant concentration of the second sample is greater than or equal to 98% of the total residual oxidant concentration of the first sample. In other embodiments, the amount of the first and/or second oxidizing biocide being added is further reduced if the total residual oxidant concentration of the second sample is about 99% to about 100% of the total residual oxidant concentration of the first sample. Still yet, in other embodiments, the amount of the first and/or second oxidizing biocide being added is further reduced if the total residual oxidant concentration of the second sample is about or in any range between about 99%, 99.1%, 99.2%, 99.3%, 99.4%, 99.5%, 99.6%, 99.7%, 99.8%, 99.9%, and 100% of the total residual oxidant concentration of the first sample.

According to one or more embodiments, methods further include measuring a free residual oxidant concentration of the second sample and reducing an amount of the first and/or second oxidizing biocide being added if the free residual oxidant concentration is greater than 0.1 mg/L as Cl₂.

In some embodiments, the RO influent has a free residual oxidant concentration less than about 0.1 mg/L as Cl₂. The amount of the first and/or second oxidizing biocide being added may be reduced if the free residual oxidant concentration of the second sample (the sample taken closer to the RO membrane) is higher than about 0.1 mg/L as Cl₂. For the first sample (taken further from the RO membrane than the second sample), a higher free residual oxidant concentration means better biocidal performance.

In some embodiments, methods include measuring a first ORP at a first location downstream of the first and second injection points but upstream of the RO membrane inlet and increasing an amount of the first and/or second oxidizing biocide being added if the first ORP is less than about 300 mV.

In embodiments, methods further include measuring a second ORP at a second location, which is downstream of the first location but upstream of the RO membrane inlet; calculating a difference in the ORP between the second and first locations; and reducing an amount of the first and/or second oxidizing biocide being added if the second ORP decreased by less than 30 mV. In other embodiments, the amount of the first and/or second oxidizing biocide being added is reduced if the second ORP is decreased by about 0 to about 30 mV. Still yet, in other embodiments, the amount of the first and/or second oxidizing biocide being added is reduced if the second ORP is decreased by about or in any range between about 0.5, 10, 15, 20, 25, and 30 mV.

In some embodiments, the second sampling location (the sampling location closest to the RO membrane) should have an ORP no greater than about 300 mV to about 350 mV. The first sampling location (further upstream of the RO membrane than the second sampling location but downstream of the biocide injection point, such as the stabilized bromine injection point) should have an ORP higher than the second ORP. It may be about 350 mV-about 400 mV or possibly greater than about 500 mV. Biocide dosages may be reduced (especially stabilized bromine biocides) if the second ORP is greater than about 300 mV-350 mV. Biocide dosages may be increased if the second ORP is low, such as below about 200 mV. If the second ORP is less than about 300 mV-about 350 mV, dosages of stabilized bromine may be increased to have a higher ORP at the first sampling location.

Reagents that include DPD are used to measure the biocide concentration by measuring both total residual oxidant (TRO) and free residual oxidant (FRO), in a colorimetric assay method. Such methods are well established in the art. The reagents of the present disclosure may comprise additional components, such as a buffering agent and/or a chelating. The buffering agent may be, for example, a phosphate and/or a carboxylic acid. The chelating agent may be, for example, ethylenediaminetetraacetic acid (EDTA).

The TRO includes total halogen, in both stabilized and free forms, from the first oxidizing biocide and second oxidizing biocides (e.g., total and free bromine and chlorine, respectively). The FRO measurement can be used to estimate total amount of the oxidizing halogen from the first oxidizing biocide (e.g., bromine), in both stabilized and free forms. Online sensors are also commercially available to measure the FRO and TRO. Thus, grab sample measurements and online sensors can be applied at various points (as shown in FIG. 1), to determine the consumption rate of the oxidizing halogens from the first and second oxidizing biocides in the system process.

The oxidizing halogen from the first oxidizing biocide (e.g., bromine) rapidly induces biocidal effects on microorganisms present in the system, while the oxidizing halogen from the second oxidizing biocide (e.g., chlorine) prevents the regrowth of these microorganisms. Measuring the biocide concentration of the first sample from a first location downstream of the first and second injection points and upstream of the RO membrane inlet ensure the decomposition of bromine is complete before RO membrane and the amount of stabilized chlorine is sufficient high to provide the biostatic effect throughout the system.

In some embodiments, the biocide concentration is quantified by TRO. For example, the TRO may be from about 0.5 to about 5 mg/L as Cl₂, such as about 1 to about 2 mg/L as Cl₂.

Yet, in further embodiments, the methods include adjusting biocide addition to maintain a measured FRO concentration of less than 1 ppm. In other embodiments, the biocide addition is adjusted to maintain a measured biocide concentration of about 0.0001 to about 1 ppm. Still yet, in other embodiments, the methods include adjusting biocide addition to maintain a measured biocide concentration about or in any range between about 0.0001, 0.001, 0.01, 0.1, and 1 ppm.

### EXAMPLES

### Example 1: Biocidal Efficacy of Stabilized Chlorine and Bromine Reagents

Four (4) water samples were used to assess biocidal efficacy of stabilized chlorine and bromine reagents. The water samples were extracted from a microelectronics plant wastewater recycle system, with two (2) being from storage tanks of UF permeate, and two (2) from storage tanks of RO permeate that would have been fed to an ultrapure water (UPW) process.

The oxidizing biocides of stabilized bromine and chlorine reagents were prepared by reacting sodium hypochlorite/sodium hypobromite with sodium sulfamate. The reagents were added to the water samples and quantified as ppm of total chlorine. Total viable count (TVC) (CFU/ml) after 24 hours was measured via the standard plate count (SPC) agar method.

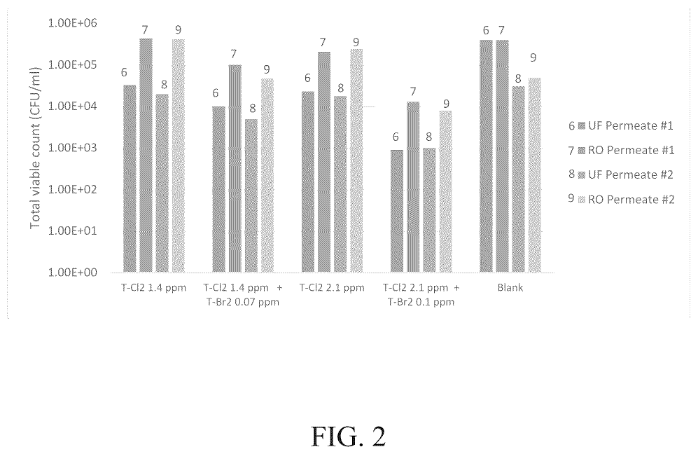

As shown in FIG. 2, the data demonstrated that stabilized chlorine reagent was largely biostatic, unable to reduce the total viable count (TVC) significantly in three (3) out of the four (4) samples. However, the stabilized bromine reagent, added in small quantities compared to stabilized chlorine reagent, had a significant effect on the biocidal efficacy. The improvement ranged from 0.52 to 0.95 log reductions for 0.07 ppm total bromine (as Cl₂) and 1.26-1.49 log reductions for 0.10 ppm total bromine (as Cl₂).

### Example 2: Biocidal Efficacy Comparison of Stabilized Chlorine vs. Stabilized Chlorine and Bromine Combination

The consumption rates of bromine and chlorine in various wastewater samples were assessed. Two (2) wastewater samples were taken from a microelectronics fabrication plant. One (1) sample was from the alkaline wastewater recycle process, and one (1) sample was from the hydrogen fluoride recycle process.

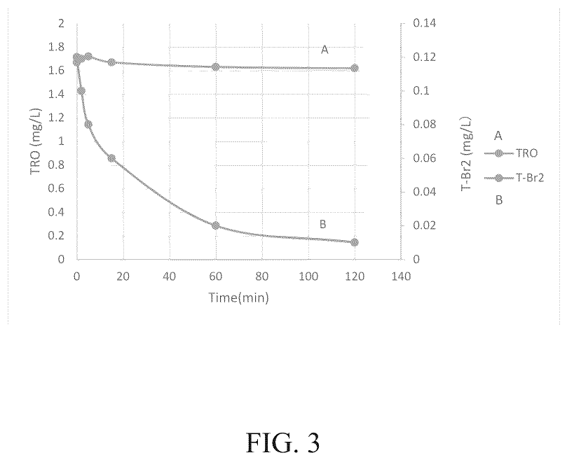

Stabilized chlorine reagents and stabilized bromine reagents were added to the water samples, with 1.7 mg/L as TRO and 0.12 mg/L as T-Br₂. The samples were periodically checked for TRO and T-Br₂ readings. As shown in FIG. 3, bromine decomposition was significantly faster than that of chlorine. Bromine was consumed in two (2) hours in one sample, and 0.5 hours in the other sample. The TRO remained relatively constant during these time periods.

### Example 3: Field Trial Results

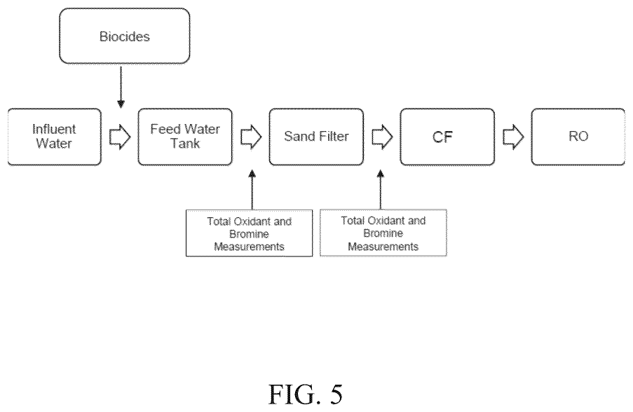

Stabilized chlorine reagents and stabilized bromine reagents were added before the feed water tank in a wastewater recycle system of a microelectronics plant, as shown in FIG. 5. The dosage was 2 mg/L as TRO and 0.1 mg/L as T-Br₂. The TRO and T-Br₂ were measured periodically at the sampling point of after feed water tank, before the sand filter (SF), and after the SF and before the cartridge filter (CF). The retention time of the feed water tank was 1.5 hours. The data showed that the total bromine remained very small (0.01-0.02 ppm) before the SF and was completely decomposed after the SF.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a biocide” is intended to include “at least one biocide” or “one or more biocides.”

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method of monitoring and controlling biocides in an aqueous medium, comprising:
adding a first oxidizing biocide at a first injection point upstream of a reverse osmosis (RO) membrane inlet;
adding a second oxidizing biocide at a second injection point upstream of the RO membrane inlet;
obtaining a first sample of the aqueous medium from a first location downstream of the first and second injection points and upstream of the RO membrane inlet;
obtaining a second sample of the aqueous medium from a second location downstream of the first location but upstream of the RO membrane inlet;
measuring a free residual oxidant concentration of the first sample and the second sample using a reagent comprising N,N-diethyl-p-phenylenediamine (DPD); and
adjusting addition of the first and/or second oxidizing biocide so that the free residual oxidant concentration in the second sample is less than about 0.1 mg/L as Cl₂ and the free residual oxidant concentration in the first sample is higher than about 0.1 mg/L as Cl₂.

2. The method of claim 1, wherein the reagent further comprises a buffering agent, a chelating agent, or any combination thereof.

3. The method of claim 1, wherein the first and second oxidizing biocides are selected from the group consisting of chlorosulfamate, bromosulfamate, iodosulfamate, urea, a stabilized chlorine or a stabilized bromine stabilized by ammonia, ethanolamine, or organic sulfamate, and any combination thereof.

4. The method of claim 3, wherein the first oxidizing biocide is the stabilized bromine.

5. The method of claim 3, wherein the second oxidizing biocide is the stabilized chlorine.

6. The method of claim 1, further comprising adding a third biocide at a third injection point upstream of the RO membrane inlet.

7. The method of claim 1, wherein the first oxidizing biocide is added simultaneously with the second oxidizing biocide, sequentially with the second oxidizing biocide, continuously, and/or intermittently.

8. The method of claim 1, wherein the first and second injection points are at substantially the same location, or wherein the first injection point is further from the RO membrane than the second injection point, or wherein the second injection point is further from the RO membrane than the first injection point.

9. The method of claim 1, wherein the first oxidizing biocide comprises from about 1 mol % to about 50 mol % of a total amount of biocide added.

10. The method of claim 1, wherein the second oxidizing biocide comprises from about 50 mol % to about 99 mol % of a total amount of biocide added.

11. The method of claim 1, wherein the free residual oxidant concentration in the second sample is less than about 0.05 mg/L as Cl₂.

12. The method of claim 1, wherein the aqueous medium comprises wastewater.

13. The method of claim 1, further comprising consuming the first oxidizing biocide before it reaches the RO membrane inlet.

14. A method of monitoring and controlling biocides in an aqueous medium, comprising:
adding a first oxidizing biocide at a first injection point upstream of a RO membrane inlet;
adding a second oxidizing biocide at a second injection point upstream of the RO membrane inlet;
obtaining a first sample of the aqueous medium from a first location downstream of the first and second injection points and upstream of the RO membrane inlet;
obtaining a second sample of the aqueous medium from a second location downstream of the first location and upstream of the RO membrane inlet;
measuring a total residual oxidant concentration of the first sample using a reagent comprising DPD;
measuring a total residual oxidant concentration of the second sample using the reagent comprising DPD; and
adjusting biocide addition based on a difference between the measured total residual oxidant concentration at the second location and the measured total residual oxidant concentration at the first location.

15. The method of claim 14, further comprising:
adjusting an amount of the first and/or second oxidizing biocide being added so that the total residual oxidant concentration of the second sample is lower than or equal to 98% of the total residual oxidant concentration of the first sample.

16. The method of claim 14, further comprising:
reducing an amount of the first and/or second oxidizing biocide being added if the total residual oxidant concentration is greater than 100 ppm.

17. The method of claim 14, wherein the reagent further comprises a buffering agent, a chelating agent, or any combination thereof.

18. A method of monitoring and controlling biocides in wastewater comprising:
adding a first oxidizing biocide at a first injection point upstream of a RO membrane inlet;
adding a second oxidizing biocide at a second injection point upstream of the RO membrane inlet;
measuring a first oxidation-reduction potential (ORP) at a first location downstream of the first and second injection points but upstream of the RO membrane inlet; and
adjusting an amount of the first and/or second biocide being added to maintain the first ORP higher than about 300 mV.

19. The method of claim 18, further comprising measuring a second ORP at a second location, which is downstream of the first location but upstream of the RO membrane inlet;
calculating a difference in the ORP between the second and first locations; and
adjusting an amount of the first and/or second oxidizing biocide being added to maintain the first ORP at least about 30 mV higher than the second ORP.

20. The method of claim 18, wherein the first oxidizing biocide is a stabilized bromine and the second oxidizing biocide is a stabilized chlorine.
